(19) United States
(12) Patent Application Publication
(10) Pub. No.: US 2026/0069490 A1
(43) Pub. Date: Mar. 12, 2026

(54) ROLLER DEVICE FOR FIDGETING AND/OR MASSAGING

(71) Applicant: Ari Horowitz, Brooklyn, NY (US)

(72) Inventor: Ari Horowitz, Brooklyn, NY (US)

(21) Appl. No.: 18/827,431

(22) Filed: Sep. 6, 2024

(51) Int. Cl. A61H 15/00; A61M 21/00; A61M 21/02

(52) U.S. Cl. CPC A61H 15/0092; A61M 21/02; A61H 2015/0042; A61H 2201/1253; A61H 2205/065; A61M 2021/0022

(57) ABSTRACT

A roller device for fidgeting and/or massaging comprising a rolling sphere located between two or more surfaces including at least a top surface and a bottom surface, the top surface having an opening for a portion of the rolling sphere to be accessible from the top surface, the bottom surface having an opening for a portion of the rolling sphere to be accessible from the bottom surface, whereby a user can roll the rolling sphere on a surface of something other than the roller device.

### BACKGROUND OF THE INVENTION

The background of the invention is in fidget and massage devices.

### SUMMARY OF THE INVENTION

In one embodiment, the invention is a roller device comprising: a rolling/rotating sphere/ball juxtaposed (alternatively placed, suspended, positioned, nested, fitted, secured, held, depending on desired function) between two or more surfaces, for example a top surface and a bottom surface and any number of surfaces in between, these surfaces can be attached to each other, or fitted into each other (e.g., permanently with glue, reversibly with screws, etc.). When only a top surface and bottom surface are present, the top surface having an opening for a portion of the rolling sphere to be accessible from (for the sphere to extend beyond) the top surface, the bottom surface having an opening for a portion of the rolling sphere to be accessible from (for the sphere to extend beyond) the bottom surface. When additional surfaces are present, they have openings for the sphere too. A case is thereby formed from a top surface, a bottom surface and optionally any number of surfaces in between, whereby a user can roll/rotate the rolling sphere. The design of the case, its number of surfaces, the way the sphere is held, etc. can influence the ease of rolling of the sphere.

An alternative case can be formed from attaching pieces side to side. The case can comprise two or more pieces, two are shown in FIGS. 21 and 22, and can be attached, by way of non-limiting example, by being fitted together, interlocking pieces, glued, screwed, etc. The case can have a rolling/rotating sphere/ball juxtaposed (alternatively placed, suspended, positioned, nested, fitted, secured, held, depending on desired function) between the pieces. Generally, the top surface having an opening for a portion of the rolling sphere to be accessible from (for the sphere to extend beyond) the top surface, the bottom surface having an opening for a portion of the rolling sphere to be accessible from (for the sphere to extend beyond) the bottom surface, whereby a user can roll/rotate the rolling sphere.

The roller device can be held in a user's palm and a user can roll/rotate the rolling sphere. The user can roll/rotate the rolling sphere with one or more digits (of a user). The user can roll/rotate the rolling sphere along a surface, along a surface of a body, etc. The roller device can be on any surface and a user can roll/rotate the rolling/rotating sphere. The user can roll/rotate the portion of the sphere accessible from the top or the bottom surface of the roller device.

In another embodiment of the roller device, the top surface has a concave shape, complimenting the bottom surface which has a convex shape, whereby the roller device sits comfortably in a user's hand. Alternatively, the top surface can be flat, convex and/or have texture/bumps on it. Additionally, the bottom surface can be flat, concave and/or have texture/bumps on it.

In another embodiment, the roller device further comprises complimentary screw threads in the top surface and the bottom surface where the top surface is fitted into the bottom surface.

In another embodiment of the roller device, the portion of the rolling sphere accessible from the bottom surface is less than the portion of the rolling sphere accessible from the top surface.

In another embodiment, the roller device further comprises at least one first low friction ring around a portion of the rolling sphere located beneath the top surface.

In another embodiment, the roller device further comprises at least one second low friction ring around a portion of the rolling sphere located above the bottom surface.

In another embodiment, the invention is a roller device comprising: at least two rolling/rotating spheres/balls juxtaposed (alternatively placed, suspended, positioned, nested, fitted, secured, held, depending on desired function) between a top surface and a bottom surface, the top surface being fitted into the bottom surface, the top surface having an opening for a portion of at least one of the at least two rolling spheres to be accessible from (for the sphere to extend beyond) the top surface (the top surface can have separate openings for each of the at least two rolling spheres), the bottom surface having an opening for a portion of at least one of the at least two rolling spheres to be accessible from (for the sphere to extend beyond) the bottom surface (the bottom surface can have separate openings for each of the at least two rolling spheres), whereby a user can roll/rotate at least one of the at least two rolling spheres. The roller device can be held in a user's palm and a user can roll/rotate at least one of the at least two rolling spheres. The user can roll/rotate at least one of the at least two rolling spheres with one or more digits (of a user). The user can roll/rotate at least one of the at least two rolling spheres along a surface, along a surface of a body, etc. The roller device can be on any surface and a user can roll/rotate the at least one of the at least two rolling/rotating spheres. The user can roll/rotate the portion of the at least one of the at least two rolling spheres accessible from the top or the bottom surface of the roller device.

In another embodiment of the roller device, the top surface has a concave shape, it can be flat, complimenting the bottom surface which has a convex shape, whereby the roller device sits comfortably in a user's hand.

In another embodiment, the roller device further comprises complimentary screw threads in the top surface and the bottom surface where the top surface is fitted into the bottom surface.

In another embodiment of the roller device, the portion of the at least one of the at least two rolling spheres accessible from the bottom surface is less than the portion of the at least one of the at least two rolling spheres accessible from the top surface.

In another embodiment, the roller device further comprises at least one first plastic ring around a portion of the at least one of the at least two rolling spheres located beneath the top surface.

In another embodiment, the roller device further comprises at least one second low friction ring around a portion of the at least one of the at least two rolling spheres located above the bottom surface.

### BRIEF DESCRIPTION OF DRAWINGS

FIG. 1 is a top plan view of an embodiment of the invention;

FIG. 2 is a perspective view of an embodiment of the invention;

FIG. 3 is an elevated side view of an embodiment of the invention;

FIG. 4 is a bottom plan view of an embodiment of the invention;

FIG. 5 is an exploded side view of the separate components of an embodiment of the invention;

FIG. 6 is an exploded perspective top view of the separate components of an embodiment of the invention;

FIG. 7 is an elevated side view of an embodiment of the invention showing line A-A;

FIG. 8 is a cross-sectional view of an embodiment of the invention along line A-A of FIG. 7;

FIG. 9 is an exploded perspective top view of the separate components of an embodiment of the invention along with a tool for opening the inventive device;

FIG. 10 shows the tool of FIG. 9 before use;

FIG. 11 is a bottom perspective view of an embodiment of the invention;

FIG. 12 is a top perspective view of an embodiment of the invention;

FIG. 13 is a side view of an embodiment of the invention;

FIG. 14 is a bottom view of an embodiment of the invention;

FIG. 15 is an embodiment of an optional key ring attachment with a conventional key ring and a stretchy material to attach to the inventive roller device (the stretchy material stretches to fit around the perimeter of the inventive roller device so it can be carried on the key ring and then it stretches for removal from the roller device), as an alternative, the material does not need to be stretchy, instead it can be a strap that is attached around the inventive roller device with a fastening device such as a snap, buckle, Velcro® type hook and loop fasteners, etc.;

FIG. 16 is an embodiment of the optional key ring attachment of FIG. 15 on an embodiment of the roller device;

FIG. 17 is a top perspective view of an embodiment of the invention;

FIG. 18 is an exploded side view of the separate components of an embodiment of the invention;

FIG. 19 is a cross-sectional view of an embodiment of the invention;

FIG. 20 includes several views on an embodiment with more than one sphere;

FIG. 21 is a perspective top view of an embodiment of a case fitted together side-by-side instead of horizontally and showing marks on top where it is fitted together; and

FIG. 22 is an exploded partial cross-sectional view of FIG. 21.

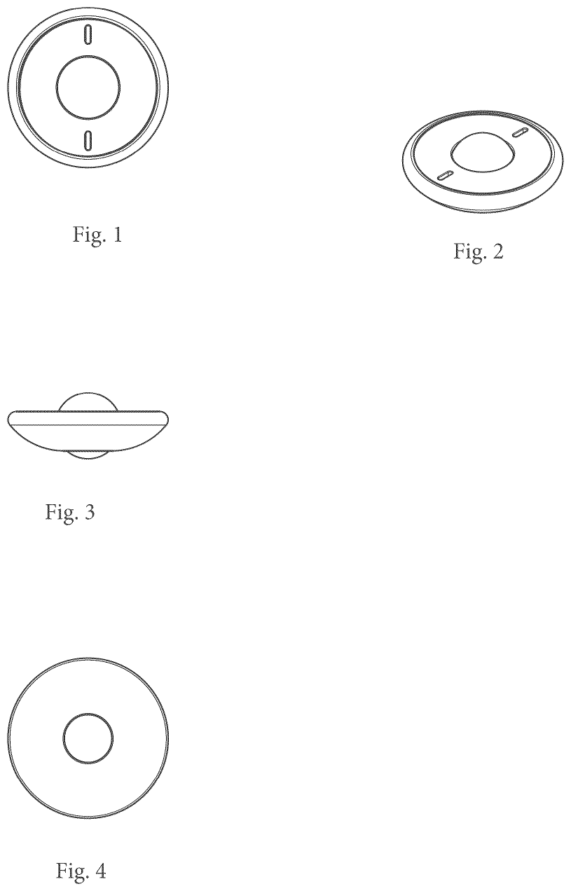

### DETAILED DESCRIPTION OF THE INVENTION

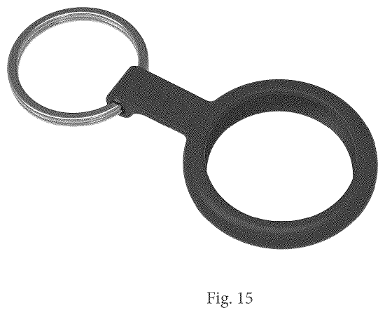

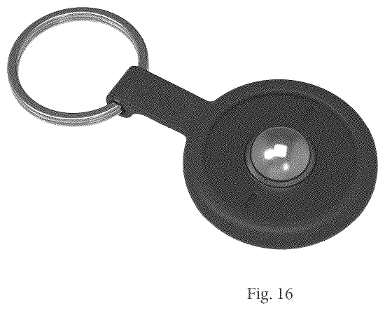

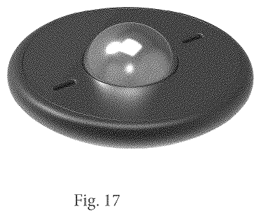

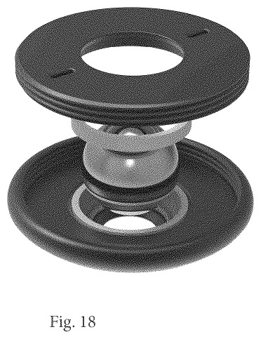

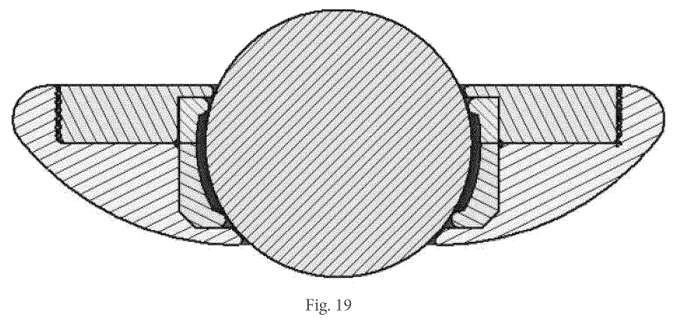

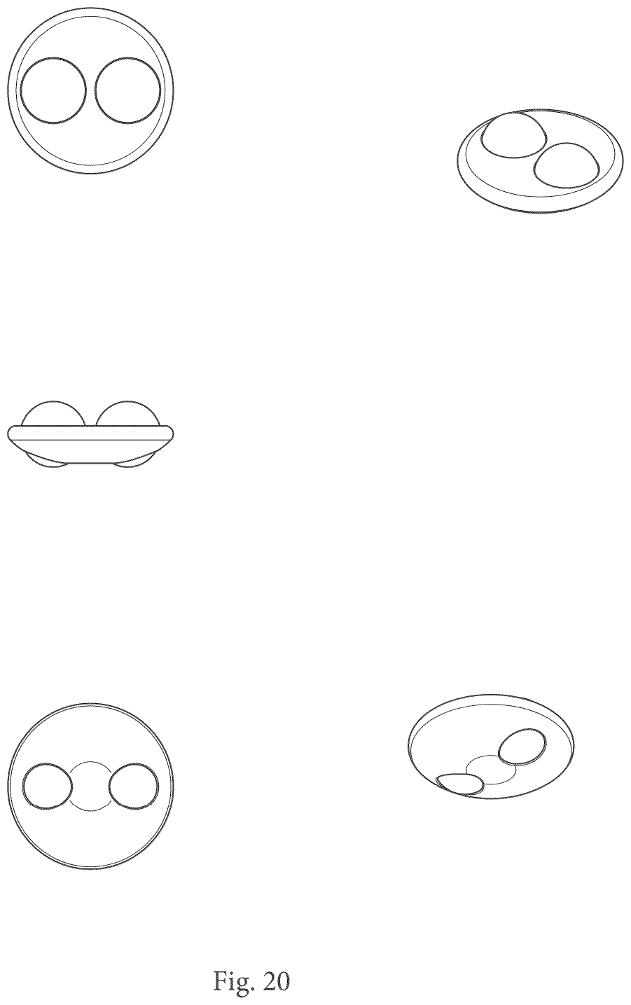

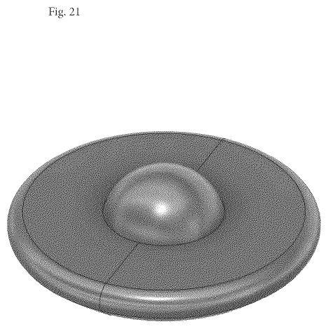

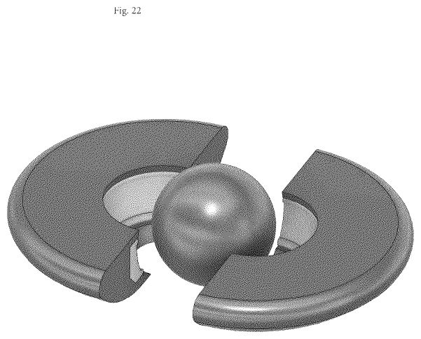

In one embodiment, the invention includes a sphere which can be rotated by a user and it can have a low (even a very low) friction, while also being silent. The rolling motion can be similar to that of swiping a smartphone. There is an optional keychain attachment, as shown in FIGS. 15 and 16.

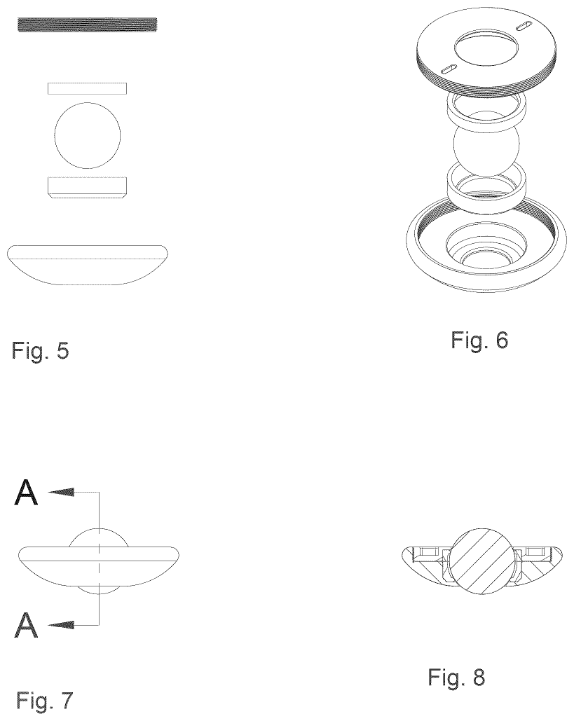

The invention can have one or more friction-reducing rings around the ball/sphere to more securely hold it in place and allow it to roll with lower friction, as shown in FIGS. 5, 6, 9 and 18. These rings can be made of ceramic, plastic, PTFE and the like.

In one embodiment of the invention, more of the ball extends beyond the top surface than extends beyond the bottom surface. This allows more of the ball on the top to be accessible based on the shape. It allows the user to turn over the roller device and rub their finger against the curved bottom surface and onto the ball, without the ball sticking out too much. This adds a different tactile feeling whichever side is up (whichever side the user interacts with).

In one embodiment, the main body is curved at the bottom so that it sits comfortably in a user's hand, and the top is flat so that the user can move a finger along the surface and around the ball. It is not necessary that a portion of the ball be exposed on the bottom.

Some of the parts shown in FIG. 6 include:
1. Outer body
2. Screw on top
3. Ball
4. PTFE top ring to hold the ball
5. PTFE bottom ring to hold the ball
   In one example of FIG. 6, the ball is a ball bearing and the rings are ceramic.

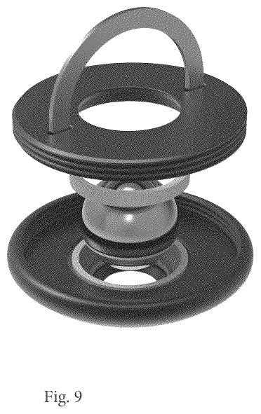

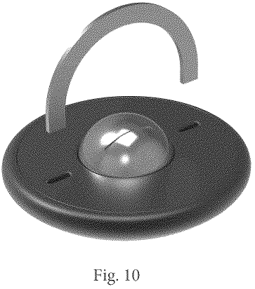

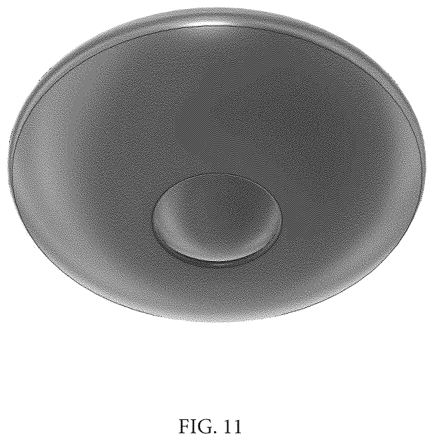

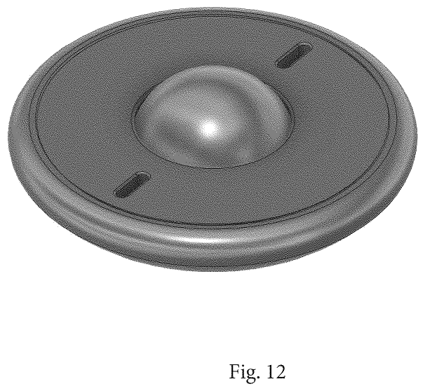

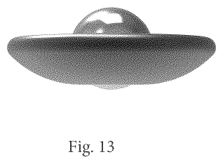

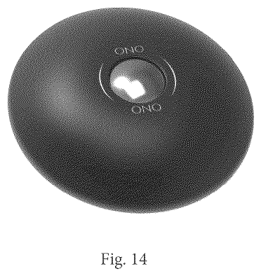

An inventive tool shown in FIGS. 9 and 10 can go into the slits in the top surface and make it easy to remove the top surface for repair, cleaning, etc.

Although certain embodiments of the present invention have been described it will be understood by those skilled in the art that the present invention should not be limited to the described embodiments. Rather, various changes and modifications can be made within the spirit and scope of the present invention as part thereof.

1. A roller device comprising: a rolling sphere juxtaposed between two or more surfaces including at least a top surface and a bottom surface, the top surface being attached to the bottom surface, the top surface having an opening for a portion of the rolling sphere to be accessible from the top surface, the bottom surface having an opening for a portion of the rolling sphere to be accessible from the bottom surface, whereby a user can roll the rolling sphere along a surface, with one or more digits, on as surface of a body.

2. The roller device of claim 1, further comprising at least one more surface between the top surface and the bottom surface.

3. The roller device of claim 1, wherein the top surface has a concave shape complimenting the bottom surface which has a convex shape, whereby the roller device sits comfortably in a user's hand.

4. The roller device of claim 1, further comprising complimentary screw threads in the top surface and the bottom surface where the top surface is fitted into the bottom surface.

5. The roller device of claim 1, wherein the portion of the rolling sphere accessible from the bottom surface is less than the portion of the rolling sphere accessible from the top surface.

6. The roller device of claim 1, further comprising at least one first low friction ring around a portion of the rolling sphere located beneath the top surface.

7. The roller device of claim 1, further comprising at least one second low friction ring around a portion of the rolling sphere located above the bottom surface.

8. A roller device comprising: at least two rolling spheres juxtaposed between a top surface and a bottom surface, the top surface being fitted into the bottom surface, the top surface having an opening for a portion of at least one of the at least two rolling spheres to be accessible from the top surface, the bottom surface having an opening for a portion of at least one of the at least two rolling spheres to be accessible from the bottom surface, whereby a user can roll the rolling sphere.

9. The roller device of claim 8, wherein the top surface has a concave shape complimenting the bottom surface which has a convex shape, whereby the roller device sits comfortably in a user's hand.

10. The roller device of claim 8, further comprising complimentary screw threads in the top surface and the bottom surface where the top surface is fitted into the bottom surface.

11. The roller device of claim 8, wherein the portion of the at least one of the at least two rolling spheres accessible from the bottom surface is less than the portion of the at least one of the at least two rolling spheres accessible from the top surface.

12. The roller device of claim 8, further comprising at least one first friction-reducing ring around a portion of the at least one of the at least two rolling spheres located beneath the top surface.

13. The roller device of claim 8, further comprising at least one second friction-reducing ring around a portion of the at least one of the at least two rolling spheres located beneath the bottom surface.
